(19) United States
(12) Patent Application Publication
(10) Pub. No.: US 2026/0042989 A1
(43) Pub. Date: Feb. 12, 2026

(54) SINGLE-USE BAG HOLDER FOR USE WITH BUILT-IN UNITS FOR BOARDS

(71) Applicant: Sartorius Stedim Systems GmbH, Guxhagen (DE)

(72) Inventor: Hannes Schmid, Felsberg (DE)

(73) Assignee: Sartorius Stedim Systems GmbH, Guxhagen (DE)

(21) Appl. No.: 19/100,405

(22) PCT Filed: Jul. 25, 2023
(86) PCT No.: PCT/EP2023/070494

§ 371 Date: Jan. 31, 2025

(30) Foreign Application Priority Data
Aug. 1, 2022 (EP) 22188086.7

(51) Int. Cl. C12M 3/00; C12M 1/00; C12M 1/34

(52) U.S. Cl. CPC C12M 23/48; C12M 21/02; C12M 23/22; C12M 23/26; C12M 23/28; C12M 31/10; C12M 41/12

(57) ABSTRACT

The present invention relates to a device and its use for accommodating a single-use container, comprising an accommodating container having an interior wall that delimits a container interior of the accommodating container for accommodating the single-use container, a plurality of grooves for accommodating electric functional elements being provided in the accommodating container interior wall.

The invention relates to a device and a method for accommodating a single-use container.

Bioreactors and Palletanks serve as a device for accommodating, storing and/or cultivating biological media such as fluids and/or cell cultures. Biological media can be provided in single-use containers that can have a volume of a few liters up to several thousand liters. The biological media are introduced into the bioreactor within a single-use container, in which they are kept at a predetermined temperature over a predetermined period of time of usually several hours. Furthermore, various examinations can be carried out on the biological medium in such a bioreactor. A bioreactor can be handled under clean room conditions, so that particularly high demands are placed on the quality assurance of the bioreactor.

So-called single-use processes are widespread in the pharmaceutical industry by now. Single-use bags in particular can be used as single-use containers in single-use processes. Both in the upstream and downstream, the use of single-use containers has proven to be time and cost saving as well as safe in terms of process technology. While there is now experience with cell culture processes and microbial cultures, processes with phototrophic organisms (algae, plant cell cultures) have so far only been realized to a limited extent. This applies in particular to a production scale of 50 liters or more.

It is therefore the object of the invention to simplify the cultivation of phototrophic organisms, in particular on a larger production scale, and in particular to improve the control of production parameters. This object is achieved by a device and a method according to the independent claims. Preferred embodiments are the subject of the dependent claims.

One aspect thus relates to a device for accommodating a single-use container, comprising an accommodating container with an accommodating container inner wall, which delimits a container interior of the accommodating container for accommodating the single-use container, wherein a plurality of grooves or channels for accommodating electrical functional elements are provided in the accommodating container inner wall. Temperature control elements, but in particular lighting units (e.g. LEDs), can be used as such electrical functional elements.

The device can be designed as a bioreactor and/or Palletank, which can be designed to accommodate single-use containers with a volume of approx. 1 liter to approx. 5000 liters, preferably with a volume of approx. 50 liters to approx. 2000 liters, particularly preferably at least approx. 500 liters. The device can be designed as a system with several components, in particular with or without a single-use container as an associated component. The device can in particular be designed to accommodate single-use containers in which there is a biological medium, such as a fluid, which is to be stored, temperature-controlled, cultivated and/or otherwise examined in the system over a predetermined period of time. The biological medium can be designed as a cell culture and/or contain phototrophic organisms.

The single-use container can be designed as a single-use bag with flexible bag walls, in particular with transparent plastic walls. The single-use container can have stirring components in its interior, which can be coupled to a stirring device of the device in order to allow the contents of the single-use container to be mixed.

The accommodating container of the device provides the container interior that is designed to accommodate the single-use container. Here, the container interior can be designed to accommodate a predeterminable type of single-use container, for example a single-use container from a predeterminable manufacturer and/or with a predeterminable filling volume. The accommodating container inner wall or walls define the container interior. Here, the accommodating container inner walls do not have to completely surround and/or delimit the container interior. For example, the accommodating container can have a stirring opening through which a stirring device can be connected to the single-use container arranged in the container interior. Such a stirring device is preferably formed at the upper end of the accommodating container. The accommodating container can thus in particular be designed without a lid and/or to be open at the top.

In the context of this invention, the terms “top”, “bottom”, “side”, “vertical”, “horizontal”, “height”, “lateral” etc. refer to the Earth's reference system in which the system or the device is arranged in an operating position.

The single-use container is preferably inserted into the accommodating container such that it rests on a bottom of the accommodating container and is in direct, physical contact with the inner walls of the accommodating container, in particular in contact with the bottom of the accommodating container and/or the inner walls of the accommodating container adjacent to the bottom. The accommodating container can have at least one openable and closable door through which the single-use container can be inserted into the container interior.

The possibility of accommodating electrical functional elements in grooves in the accommodating container inner wall improves their thermal coupling to the accommodating container inner wall and at the same time the container interior remains unimpaired or less impaired by protruding functional elements that could also impair or damage the single-use container.

Particularly preferably, lighting units for illuminating the container interior are at least partially mountable or mounted in the plurality of grooves. In this case, the functional elements are or comprise lighting units. In particular, all grooves could be designed to accommodate lighting units for illuminating the container interior. In one embodiment, lighting units could even be mounted in all grooves. In another preferred embodiment, lighting units are or will be mounted or mountable only in some of the grooves, while other grooves are provided or used for other functional elements, i.e. in particular can be equipped or are equipped with other functional elements.

The lighting units arranged in the grooves are preferably arranged such that they emit the light output at least predominantly in the direction of the container interior, i.e. to the single-use container or the medium contained therein. In the operating posture or position of the device, a single-use container is inserted into the device. The single-use container does not have to be a component of the device. However, the single-use container can be designed as a component of the device. The lighting units can be arranged in the container interior such that they exchange heat with the preferably temperature-controlled accommodating container inner wall. In this case, the lighting units in particular can preferably be temperature-controlled, e.g. cooled, using a temperature-control unit described later. This enables heat dissipation of waste heat that can arise during operation of the lighting units. The lighting units do not require their own cooling, but can be temperature-controlled or cooled using the device's temperature-control unit.

The device is particularly suitable for accommodating single-use containers with phototrophic organisms or cell cultures. The medium in the single-use container can be irradiated using the light emitted by the lighting units. Here, the walls of the single-use container are preferably transparent to at least the light wavelength(s) in which the lighting units emit light into the container interior. Thus, in a further aspect, the invention relates to the use of a device according to the invention for processing phototrophic organisms. The device is preferably used for cultivating mosses in a process for producing alpha-galactosidase.

In the context of this invention, the term “light” does not necessarily mean visible light, but can refer to electromagnetic radiation with a wavelength of about 50 nm to about 50 μm. The term therefore includes ultraviolet and infrared light in particular. The wavelength or wavelength range of the light emitted by the lighting units can be tailored to specific needs of the medium in the single-use container. The lighting units can each have one or more light sources that are aligned so as to emit light in the direction from the accommodating container inner wall to the container interior, in particular in a direction toward the spatial region in which the single-use container is arranged in the operating position, e.g. toward a center point and/or a central axis of the container interior.

The device can also have a control system that serves to control the light output by the lighting units. The lighting units can e.g. emit modulated and/or non-modulated electromagnetic radiation, or also coherent electromagnetic radiation such as laser light. The lighting units can preferably be controlled so as to emit a controllable amount of light during controllable periods of time. In particular, the lighting units can also be switched off. According to one embodiment, one (or each) lighting unit has a plurality of light sources arranged within the same groove. The plurality of light sources of a lighting unit are preferably carried by a common carrier, via which the respective lighting unit is arranged within the groove on the accommodating container inner wall. A substrate can serve as the carrier, in particular a substrate that is thermally conductive at least partially, whereby heat developed at the light source(s) can be dissipated very efficiently to the container inner wall. As will be described in more detail later, it is particularly preferred if the outside of the container inner wall is temperature-controlled using a temperature-control unit (e.g. a temperature-control medium) that controls the temperature of the container inner wall on the outside.

In one embodiment, the lighting units in the grooves are at least partially covered or cast in by a (translucent or transparent) cast material (e.g. a translucent or transparent resin or lacquer layer). This cast material supports a mechanically stable fastening of the respective lighting unit in the corresponding groove and at the same time protects the lighting units and the single-use container from mutual damage. It particularly preferably allows at least partial refractive index adjustment for light to be radiated from the lighting unit into the single-use container, whereby as high a proportion of the light from the lighting unit (in particular from the light sources) as possible can be coupled into the single-use container in a homogeneous distribution. An epoxy resin can be used as the cast material. The resin layer and/or lacquer layer is particularly transparent for the wavelength(s) emitted by the lighting units. The cast material particularly preferably provides a hermetic seal (IP 65) of the electrical functional units (and their direct electrical terminals) in the grooves from the environment.

In one embodiment, the grooves with the lighting units mounted therein are closed off from the container interior using (translucent or transparent) groove covers such that the inner surfaces of the (translucent or transparent) groove covers together with the inner surfaces of the accommodating container inner wall formed between the grooves, in particular adjacent to the grooves, form continuous surfaces, i.e. in particular without steps. The thus-resulting continuous accommodating container inner surface preferably does not have any (in particular convex) edges. This actually has several advantages. Firstly, the interior of the accommodating container is easy and thorough to clean. Furthermore, possible mechanical damage or stress on the single-use container is avoided or reduced. In addition, this also ensures consistently good contact between the single-use container (and thus the medium contained therein) and the accommodating container. This consistently good, homogeneous contact improves both the thermal and optical coupling of the single-use container (and thus its contents) to the accommodating containers. This leads to improved thermal power transfer (e.g. cooling and/or heating power) and thus to a more reliable temperature control even for large process volumes. In particular, local temperature peaks are also avoided, even if lighting units in the grooves locally generate heat. The good optical coupling ensures the highest possible spatial and spectral homogeneity of illumination of a medium contained in the single-use container by the lighting units contained in the grooves. This in turn also improves process quality and efficiency, especially for large process volumes.

These or some of these advantages are achieved particularly effectively if a gap (air gap) is formed in the respective groove below the groove cover, in particular between the lighting unit and the groove cover, preferably immediately below the groove cover. The groove covers are preferably designed as disks with a substantially homogeneous thickness. Unlike without such groove covers, when groove covers are used, it can be advantageous either not to fill the grooves at all with a cast material as already described or at least to provide such a gap between the cast material and the groove cover. An air gap is therefore preferably formed within the grooves at least between the lighting unit and the respective groove cover, which air gap is in particular large enough to keep interference of the radiation generated by the lighting units within the air gap by the surfaces delimiting the air gap to a minimum. For example, an air gap with a thickness in the range of approx. 0.5 mm to approx. 5 mm can be provided. The air gaps form a controllable and stable optical transition between the lighting units and the groove covers. It was found that in the case of direct (mechanical) contact between the groove covers and the lighting units (or a cast material covering the lighting units), the optical quality (in particular the intensity and homogeneity) of the incident light changes over time, in particular deteriorates. In the context of this development, this was attributed to a poorly controllable detachment effect between the lighting units (or a cast material) and the groove covers, e.g. due to thermal and/or mechanical stresses, which is avoided by providing an air gap.

Preferably, each of the plurality of grooves has a two-step cross-section on both sides perpendicular to a longitudinal axis of the respective groove such that on both sides of a central groove channel, a support surface is set back by a first step relative to the inner surface of the accommodating container inner wall and a groove base of the central groove channel is set back by a second step relative to the two support surfaces. In other words, beside a central groove channel (for accommodating the at least one lighting unit), each groove comprises a groove shoulder on both sides. The support surfaces formed by the groove shoulders on both sides serve in particular to accommodate the groove cover. To this end, the first step in particular has a height that substantially corresponds (i.e. apart from a possible thickness of an additional adhesive layer) to a thickness of the respective groove cover. The second step in particular has a height that is sufficient to accommodate the (respective) lighting unit within the central groove channel (under the groove cover).

Depending on the embodiment, the height of the first step is, for example, in a range from about 0.5 mm to about 5 mm, preferably in a range from about 1 mm to about 3 mm. Alternatively or additionally, the height of the second step is, for example, in a range from about 2 mm to about 15 mm, preferably in a range from about 2 mm to about 10 mm, even more preferably in a range from about 3 mm to about 5 mm. Alternatively or additionally, a width of the respective support surface (shoulder width) is, for example, in a range from about 1 mm to about 5 mm, preferably in a range from about 1 mm to about 3 mm. Alternatively or additionally, the width of the central groove channel (perpendicular to its longitudinal extension) is, for example, in a range of about 5 mm to about 30 mm, preferably in a range of about 10 mm to about 20 mm. This makes it easy to lay LED strips in the grooves, for example. The lighting units that can be mounted or are mounted in the grooves therefore comprise LEDs in particular as light sources. This means that both modulated and unmodulated radiation can be emitted. LEDs are suitable as particularly efficient light sources because LEDs generate relatively little heat in relation to the amount of light emitted, which can reduce heat generation and at the same time increase the emitted light output. Furthermore, LEDs can be designed with a low construction height, so that these light sources can be completely accommodated within the accommodating container inner wall even with a comparatively small groove depth.

In one embodiment, the lighting units that are mountable or mounted in the grooves are designed to emit light substantially in particular in a selected spectral range within a wavelength range of about 50 nm to about 50 μm (UV to IR). The lighting units preferably emit at least 90% of the emitted light power with a predetermined wavelength or a predetermined wavelength spectrum within a range between 50 nm and 50 μm. The lighting units can thus be designed to emit electromagnetic radiation with the predetermined wavelength or a predetermined wavelength spectrum. This can in particular be laser light and/or light from LEDs. The predetermined wavelength or the predetermined wavelength spectrum can be matched to the contents of the single-use container, in particular to wavelengths that influence the metabolism of the cells located therein. Here, the lighting units can e.g. be designed to emit at least 90% of the emitted light power at the predetermined wavelength or in the predetermined wavelength spectrum. The lighting units can also form light source groups that emit light at different wavelengths and can preferably be controlled selectively in order to change the spectral distribution of the light radiated into the container interior.

In one embodiment, a plurality of grooves in the accommodating container inner wall extend in a ring shape and/or parallel to one another, in particular horizontally in an operating position of the device. Thus, the grooves extend substantially horizontally in their longitudinal extension at least in the region of side wall portions of the accommodating container inner wall in the operating posture or position of the device, i.e. in particular in a plane perpendicular to a (virtual) cylinder axis of cylinder casing segments of the side wall portions. The horizontal course of the grooves allows targeted lighting only in the region of the filling, even with different fill levels inside the container (or with single-use containers of different heights), by activating lighting units in individual grooves up to the fill level and deactivating them above the fill level. This avoids drying out and burning in of cell material adhering to the single-use container above the liquid level. In this variant, the accommodating container inner wall can also be prefabricated partially with the grooves in a planar manner and then deformed according to the desired cylinder curvature. This has the additional advantage that the accommodating container inner wall, which has been prefabricated in a planar manner with the grooves, can then be bent to be round without causing uncontrolled or angular deformations (e.g. along the course of the grooves).

In another embodiment, a plurality of the grooves extend in a straight line and/or parallel to one another and are preferably arranged equidistant from one another. On the one hand, this allows the device to be manufactured and assembled as simply as possible. On the other hand, it supports the most homogeneous illumination of the container interior. This also keeps local inhomogeneities in the heating of the accommodating container inner wall due to the operation of the lighting units to a minimum.

In one embodiment, the accommodating container inner wall at least partially forms a part of a cylinder casing surface and particularly preferably it is composed entirely or at least partially from parts of cylinder casing surfaces. The grooves in one or in each such part of a cylinder casing surface preferably extend in parallel to the respective cylinder axis. They are therefore arranged so that their longitudinal extension is parallel to the axial direction of the respective cylinder casing surface. This can have manufacturing advantages in particular. For example, LED light strips can be installed very easily within the straight grooves. The groove covers can also be easily manufactured and assembled using substantially planar plates (e.g. made of glass or plexiglass), whereby mechanical stresses are avoided. In this way, it is also relatively easy to initially manufacture the accommodating container inner wall in a planar manner with a large number of parallel grooves in order to then curve individual portions around an axis (cylinder axis) parallel to the groove course.

Preferably, the accommodating container inner wall comprises at least partially metal or it is at least partially made of metal, with aluminum and/or stainless steel being used as the metal here. This is particularly preferred with regard to the thermal conductivity of metal, in particular in order to be able to efficiently dissipate the heat generated during operation of the electrical functional elements (e.g. lighting units) and/or to be able to efficiently regulate the temperature in the interior of the accommodating container.

In this context in particular, it is particularly advantageous if the device also comprises a temperature control unit for temperature control of the accommodating container inner wall. In one embodiment, the accommodating container has an accommodating container outer wall which, together with the accommodating container inner wall, forms a temperature control intermediate space which can be filled with and/or flown through by a temperature control fluid in order to control the temperature of the container interior. The at least one accommodating container inner wall can be temperature controlled using the temperature control unit. The temperature control unit can control the temperature of the accommodating container inner wall to a predeterminable, in particular to an adjustable temperature. The temperature control unit can be designed in particular to cool the accommodating container inner wall. Here, the temperature control unit can either be arranged directly on an outside of the accommodating container inner wall facing away from the container interior, or at least be in direct heat exchange with the accommodating container inner wall. The accommodating container inner wall can have a high thermal conductivity, so it can be made of metal, for example.

If lighting units are arranged in the grooves (which are open toward the container interior) of the accommodating container inner wall (which are covered at most by a translucent groove cover), the lighting units are in the immediate proximity of the single-use container in order to be able to illuminate its contents efficiently. On the other hand, they are in good heat-conducting contact with the accommodating container inner wall, which is also heat-conductive. The proximity between the single-use container and the lighting units enables particularly efficient light irradiation into the interior of the single-use container with greatly reduced losses. At the same time, temperature control with the temperature control unit can prevent the lighting units from developing too much heat, which could otherwise damage the cell cultures and damage a plastic wall of the single-use container.

The device is therefore particularly suitable for accommodating single-use containers that have a culture volume for a microbial culture, a cell culture, a culture of cell and/or tissue compounds of plant and/or animal cells and/or hybrid forms.

Preferably, a maximum cooling capacity of at least about 5 KW, preferably at least about 10 KW, more preferably at least about 15 KW, most preferably at least about 20 kW, can be achieved by the temperature control unit.

According to one embodiment, grooves are formed on at least 10%, preferably at least 20%, more preferably at least 30%, most preferably at least 40% of the region of the interior of all lateral accommodating container inner walls, in particular for accommodating lighting units. This increases the light output and/or the number of photons and the homogeneity of their distribution, which can be emitted into the container interior. One goal here can be largely uniform lighting across the outer surfaces of the container. The lateral inner walls of the accommodating container are those walls of the accommodating container that delimit the container interior in a lateral direction. For example, in the case of a substantially cylindrical, upright accommodating container, these are all walls of the cylinder casing without the cylinder base and without the cylinder cover.

According to one embodiment, in an operating posture or position of the device, the single-use container is in contact with the inner surface of the accommodating container inner wall (in the region between the grooves) and with the lighting units or the groove covers (in the region of the grooves). This enables particularly efficient or low-loss lighting and temperature control of the contents of the single-use container.

According to one embodiment, the accommodating container is designed to accommodate single-use containers with a volume of at least about 1 liter, preferably at least about 10 liters, even more preferably at least about 50 liters, further preferably at least about 100 liters, even more preferably at least about 200 liters, particularly preferably at least about 500 liters, very particularly preferably at least about 1000 liters, most preferably at least about 2000 liters. For example, the container interior has a volume in the range of about 1 liter to about 5000 liters, preferably in the range of about 50 liters to about 2000 liters. With previously known devices, it has not been possible to illuminate and cultivate single-use containers with such large volumes equally efficiently. Only the particularly efficient combination of lighting and cooling by the device according to the invention enables the particularly uniform light exposure and temperature control required for this. It should be noted here that the light emitted by the lighting units may not penetrate all the way to the middle of the medium in the single-use container, but that mixing the medium using a stirring device can still provide sufficiently uniform illumination of the medium. The possibility of embedding the lighting units in the grooves of the accommodating container inner wall also avoids mechanical stress on the single-use container and local temperature peaks on the inner edges of the accommodating container. Moreover, the accommodating container can be cleaned very easily and reliably.

According to one embodiment, the device complies with the GMP guidelines. The GMP guidelines, which are short for “Good Manufacturing Practice”, are guidelines on requirements for hygiene, premises, equipment, documentation and controls in the pharmaceutical sector. The device can be designed such that it meets the requirements specified in the GMP guidelines, in particular the requirements for sterility.

Overall, the invention offers some advantages both in production and in operation, at least in some of the embodiments described. For example, embedding the electrical functional elements (particularly lighting units) in grooves in the accommodating container inner wall allows the interior of the accommodating container to be cleaned easily and reliably. In addition, the electrical functional elements (e.g. LEDs) are well protected even in the event of careless handling by being mounted in the grooves and, in particular, by closing the grooves with groove covers. Particularly with regard to the use of groove covers, the formation of groove shoulders as support surfaces for the groove covers allows secure mounting (e.g. by gluing), as this both simplifies the attachment of the groove covers and ensures high durability in operation. But other than that, the electrical functional elements are easy to mount in the grooves and are particularly durable in operation. In addition, the risk of hot spots (temperature peaks) occurring can be reduced by avoiding direct contact between LED lighting surfaces and single-use containers. The wiring can also be laid within the grooves, at least in the region of the terminals to the electrical functional elements (e.g. LED), and preferably be cast together with the electrical functional elements, whereby the terminals can be protected very easily and reliably against mechanical or external chemical influences.

The invention will be described in more detail below using exemplary embodiments shown in figures. The same reference numerals designate the same or similar components of the embodiments. Individual features of the embodiments can be combined with other embodiments. The figures show:

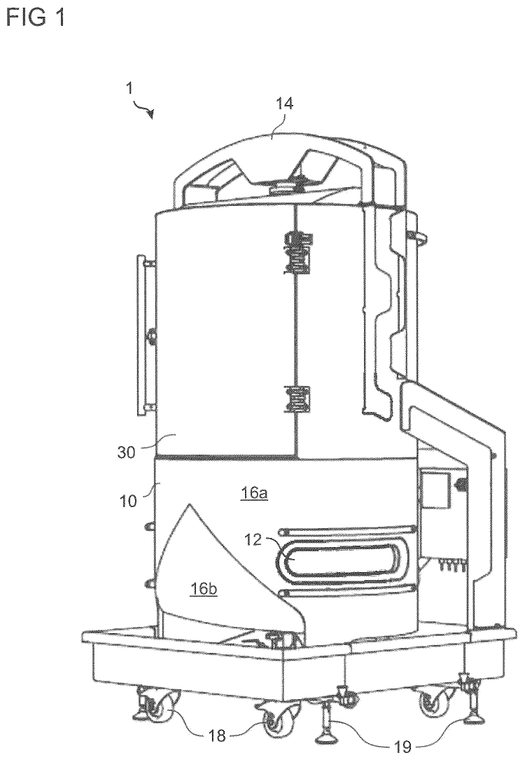

FIG. 1: in a perspective illustration, a device for accommodating a single-use bag;

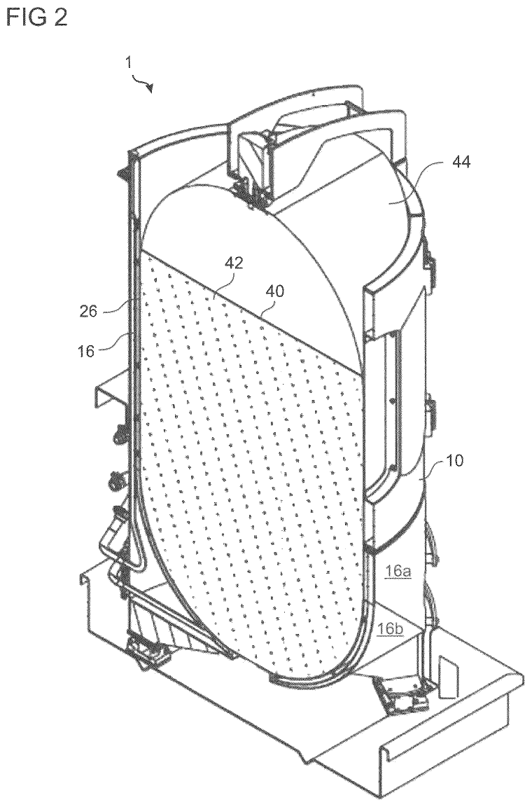

FIG. 2: in a perspective illustration, a vertical sectional view through a device for accommodating a single-use bag;

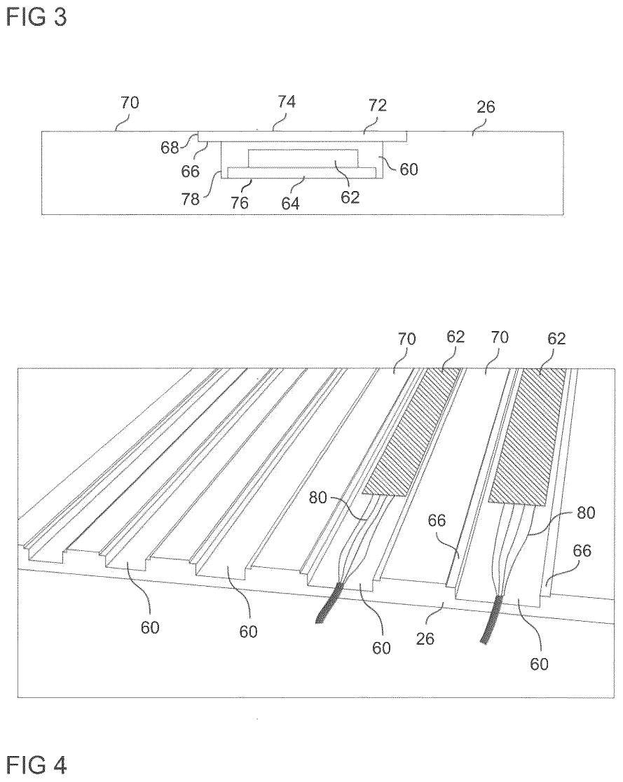

FIG. 3 in a schematic illustration, a cross-section through an accommodating container inner wall with a lighting unit housed in a groove;

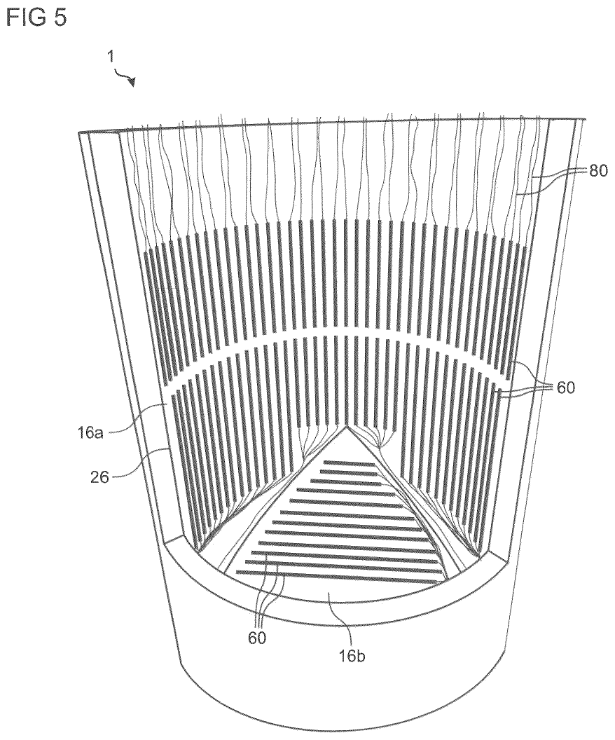

FIG. 4 in a schematic illustration, an accommodating container inner all with a plurality of parallel grooves and partially mounted lighting units;

FIG. 5 in a perspective illustration, a schematic view of an opened device for accommodating a single-use bag;

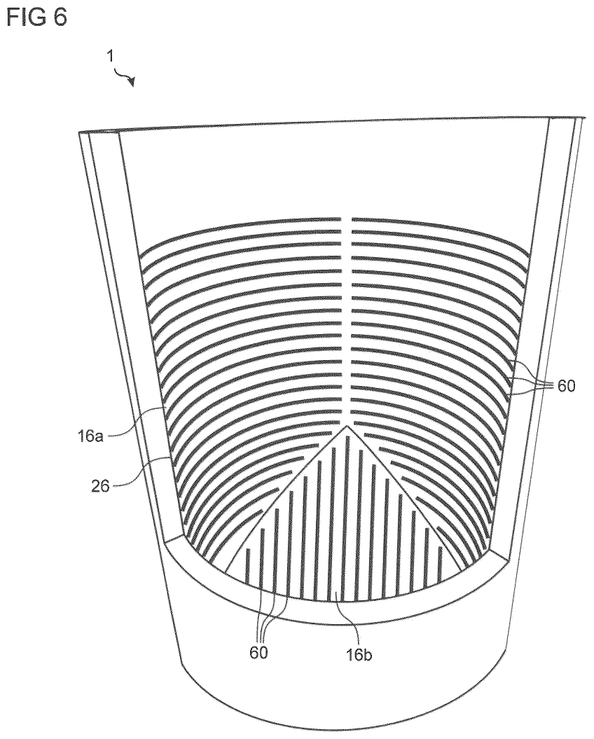

FIG. 6 in a perspective illustration, a schematic view of another opened device for accommodating a single-use bag.

FIG. 1 shows a perspective illustration of a device 1 for accommodating a single-use bag as a single-use container. The device 1 shown in the figures can be designed as a component of a system for accommodating a single-use bag. The device 1 includes an accommodating container 10 having substantially the shape of a vertically arranged cylinder, i.e. the cylinder axis of which is arranged to be substantially vertical. The accommodating container 10 includes a container interior into which a single-use bag can be inserted, which may contain a biological medium, for example. The biological medium in the single-use bag is stored and/or illuminated in the container interior of the accommodating container 10 for a predeterminable period of time. While the single-use bag with the biological medium is inside the accommodating container 10, different reactions can take place with or on the biological medium. The device 1 can therefore also be designed as a bioreactor.

To observe the biological medium, one or more viewing windows 12 are formed in a side wall portion 16a, through which one can look from the outside through the accommodating container wall into the container interior of the accommodating container 10 in order to observe the biological medium. In order to make the container interior accessible, the device preferably also includes a container door 30. The container door 30 extends in width, i.e. in the horizontal direction, for example approximately over a cylinder segment (of e.g. approx.) 100° of the accommodating container 10 and preferably also comprises part of an accommodating container inner wall in which grooves shown later are formed.

When the container door 30 is open, access to the container interior of the accommodating container 10 is possible. For example, the single-use bag can be inserted into the container interior of the accommodating container 10 through the door opening from a lateral direction, i.e. substantially in a horizontal direction of movement.

The device 1 is mounted on rollers 18 on which the device can be pushed through a room. In addition to the rollers 18, the device 1 can have fixing feet 19 at the lower end, which serve to fix and correctly align the device 1, e.g. on uneven floors.

In the embodiment shown, the accommodating container 10 is designed to be open at the top. Instead of a cylinder lid (or in a cylinder lid not shown), the accommodating container 10 has a stirring opening. Above the accommodating container 10, which is open at the top, a stirring device 14 is formed, via which a stirring rod can be connected to the single-use bag through the stirring opening so that the interior of the single-use bag can be mixed. The stirring rod can be arranged inside the single-use bag and connected to the stirring device 14 via a coupling. The stirring device 14 is formed centrally above the accommodating container 10 and is carried by a carrier bridge that rests on an upper edge of the accommodating container 10 on opposite side wall portions of the accommodating container 10.

FIG. 2 shows a perspective illustration of a vertical section through the device 1, wherein the lighting units inserted in grooves are not yet explicitly visualized in this view either. They face toward the container interior, which in this illustration is filled with a biological medium 42. FIG. 2 shows e.g. a single-use bag 44 as a single-use container, more precisely a section through this single-use bag 44, which is arranged in the container interior of the accommodating container 10. The biological medium 42, which is filled up to a predetermined fill level 40, is arranged in the container interior of the accommodating container 10 and at the same time also in the interior of the single-use bag 44. The biological medium 42 extends from the bottom of the accommodating container 10 up to the fill level 40 and thus fills in particular the entire internal volume of the accommodating container 10 up to the fill level 40, minus the volume of the walls of the single-use bag 44. The single-use bag 44 is held in shape by a container wall 16 of the accommodating container 10, which in particular comprises a bottom portion 16b and the side wall portion 16a and can extend upward from the (partially rounded) bottom portion 16b of the accommodating container 10 up to and beyond the fill level 40. The container wall 16 forms or comprises in particular an accommodating container inner wall 26, the groove structure of which will be shown in more detail later.

In the embodiment shown, the container wall 16 is composed of several portions, which in particular comprise the side wall portion 16a and a bottom portion 16b. Preferably, each of the portions is formed substantially as a segment of a cylinder casing. In the embodiment shown, for example, a (virtual) cylinder axis of the cylinder casing shape of the side wall portion 16a extends substantially vertically, while a (virtual) cylinder axis of the cylinder casing shape of the bottom portion 16b extends substantially horizontally. Particularly preferably, the grooves in the accommodating container inner walls, which will be shown in more detail later, also extend in parallel to the associated (virtual) cylinder axis of the corresponding portion. This allows the lighting units and any groove covers to be manufactured and mounted in a simple, straight line or even substantially planar manner. The container walls 16 of the accommodating container 10 are at least partially designed as a temperature control cavity wall in which a temperature control medium (not shown in the figures) flows. The temperature control medium can be regulated to a low pressure of less than 0.5 bar, or to a pressure of up to about 6 bar. The temperature control cavity wall can extend over the entire accommodating container walls 16, including the bottom portion 16b and the side wall portion 16a, from the container bottom upward to above the fill level 40 up to a predeterminable temperature control level. The temperature control level can be arranged substantially 1 cm to 20 cm vertically above the fill level 40. The accommodating container inner wall 26 provided with the grooves can be designed as the inner wall of the temperature control cavity wall or can be arranged on the inner wall of the temperature control cavity wall and thermally connected to it. In other words, the accommodating container inner wall 26 can either be manufactured separately from the temperature control cavity wall and then connected to the temperature control cavity wall or can be manufactured directly as part of the temperature control cavity wall.

The biological medium 42 can be in thermal contact with the accommodating container inner wall 26 (and thus directly or indirectly with the temperature control cavity wall) (via the bag wall of the single-use bag 44). The biological medium 42 can thus be regulated to a predeterminable temperature via the temperature control medium. The device 1 can in particular be designed and provided to control the temperature of the container interior to be a predeterminable target temperature of approx. 0° C. to approx. 80° C., preferably from approx. 20° C. to approx. 40° C. The temperature control cavity wall almost completely surrounds the container interior of the accommodating container 10 up to and beyond the fill level 40. In the embodiments shown in the figures, “almost completely surrounds” means that the temperature control cavity wall completely surrounds the container interior up to the temperature control level, except for those positions at which the viewing windows 12, if present, are arranged. Even the container door 30 can in one embodiment be designed with a temperature control cavity wall and/or with an accommodating container inner wall having grooves.

Alternatively or in addition to the temperature control cavity wall, it is also possible for temperature control elements (e.g. electrical heating and/or cooling elements) to be accommodated in at least some of the grooves in the accommodating container inner wall. In this case, not all grooves are used to accommodate lighting units, but some of them can be used to accommodate (in particular electrical) temperature control elements.

FIG. 3 shows, in a schematic illustration, a cross-section through an accommodating container inner wall 26 perpendicular to a longitudinal extension of grooves with a lighting unit 62 accommodated in a groove 60. The accommodating container inner wall 26 is shown planar in the section in FIG. 3, although in use it is preferably cylindrically curved, in particular with a cylinder axis parallel to the longitudinal direction of the groove 60 (i.e. perpendicular to the drawing plane of FIG. 3). In a preferred embodiment, the accommodating container inner wall 26 can initially be manufactured to be planar, i.e. provided with the grooves 60, before it is then preferably curved cylindrically for the manufacture or mounting of the device 1. However, since the radius of curvature of the cylindrical curvature is preferably significantly larger than the lateral dimensions of the grooves, i.e. the groove widths, the geometric relationships shown schematically will only change slightly during this deformation. However, even these minor changes can be taken into account during manufacture in order to be able to easily insert all of the components described below into the grooves. The accommodating container inner wall 26 is particularly preferably made of metal by bending and/or rolling and/or milling and/or drilling and/or 3D printing.

In the embodiment shown in FIG. 3, the lighting unit 62 comprises a plurality of light sources arranged on a common carrier 64. The light sources can be individual LEDs, for example, arranged on the carrier 64. In the embodiment shown, the grooves 60 have a groove shoulder 66 on both sides, which is set back by a first step 68 relative to an inner surface 70 of the accommodating container inner wall 26 formed between the grooves. This groove shoulder 68 serves as a support surface for a groove cover 72, which closes the groove toward the container interior such that an inner surface 74 of the groove cover 72 together with the inner surface 70 of the accommodating container inner wall 26 forms a continuous surface, in particular without edges. At least at the transition between the inner surface 74 of the groove cover 72 and the inner surface 70 of the accommodating container inner wall 26, there is preferably no edge, but preferably at least no convex edge. The height of the first step 68 is preferably in a range from about 0.5 mm to about 5 mm, preferably in a range from about 1 mm to about 3 mm. It substantially corresponds to a thickness of the groove cover 72 insofar as the continuous inner surface is thereby achieved. If the groove cover 72 is glued to the groove shoulder 66, the first shoulder 68 is preferably greater than the thickness of the groove cover 72 by the thickness of the adhesive layer. The width of the respective groove shoulder 66 is preferably in a range from about 1 mm to about 5 mm, preferably in a range from about 1 mm to about 3 mm.

In the embodiment shown, a groove base 76, as a preferably planar bottom surface of the central groove channel, is also set back by a second step 78 relative to the two groove shoulders 66. The lighting unit 60, preferably together with the carrier 64, is attached to the groove base 76. The height of the second step 78 (i.e. the depth of the groove base relative to the groove shoulders 66) is in a range from about 2 mm to about 15 mm, preferably in a range from about 2 mm to about 10 mm, even more preferably in a range from about 3 mm to about 5 mm. The height of the second step 78 is in particular at least as large as or preferably greater than a height of the lighting unit 62 (including support 64). A remaining cavity within the groove 60 is preferably filled with a cast material, so that the lighting unit 62 in the groove 60 is at least partially covered or cast in by the cast material. A width of the central groove channel (perpendicular to its longitudinal extension), i.e. in particular a width of the groove base 76, is for example in a range from about 5 mm to about 30 mm, preferably in a range from about 10 mm to about 20 mm. This makes it easy to lay LED strips in the grooves, for example.

FIG. 4 shows, in a schematic illustration, an accommodating container inner wall 26 with a plurality of parallel, in particular equidistant grooves 60 and partially mounted lighting units. This illustration is again only schematic insofar as a possible cylindrical curvature is not explicitly shown here. The grooves 60 are equipped with groove shoulders 66, similar to the illustration in FIG. 3. Lighting units 62 with associated wiring 80 are already mounted in some of the grooves 60. Before the grooves 60 are provided with groove covers, the lighting units 62, preferably with the portions of the wiring 80 extending in the grooves 60, are cast in using a casting material. The (cast) grooves are then closed with groove covers, which in particular form a continuous surface with the inner surfaces 70 of the accommodating container inner wall 26.

In principle, it is preferred if the distances between adjacent grooves 60 (distances between the mutually facing groove shoulders of adjacent grooves) are at least partially no greater than five times, preferably no greater than three times, most preferably no greater than twice the width of the grooves (including their groove shoulders). This makes it possible to achieve the most uniform lighting possible if the grooves are (at least partially) equipped with lighting units.

FIG. 5 shows, in a perspective illustration, a schematic view of an opened device 1 for accommodating a single-use bag. As shown schematically in this illustration, the accommodating container inner wall 26 can be composed of several portions, which in particular comprise a side wall portion 16a and a bottom portion 16b of the accommodating container. Preferably, all portions of the accommodating container inner wall 26 are shaped as cylinder casing segments such that a (virtual) cylinder axis of the cylinder casing segments of the side wall portions 16a is substantially vertical in the operating position of the device 1, while a (virtual) cylinder axis of the cylinder casing segments of the bottom portions 16b is substantially horizontal in the operating position of the device 1. This shape of the wall portions allows for simple manufacture while simultaneously avoiding strongly concave corners inside the accommodating container.

In particular, it is preferred if the grooves 60 in the portions of the accommodating container inner wall 26 extend substantially parallel to the respective (virtual) cylinder axis of the corresponding wall portion. The grooves 60 in the side wall portions 16a thus extend substantially vertically, while the grooves 60 in the bottom portions 16b extend substantially horizontally. As a result, the grooves 60 always extend in a straight line despite the curvature of the accommodating container inner wall 26. On the one hand, this simplifies mounting of the lighting units in the grooves 60. On the other hand, it also promotes the most homogeneous possible light distribution. This also facilitates the manufacture and mounting of transparent groove covers that extend in a straight line, preferably even in a planar manner. Finally, the manufacture of the accommodating container inner wall in this geometry is also comparatively simple. In particular, the accommodating container inner wall can be prefabricated partially with the grooves in a planar manner and then be deformed according to the desired cylinder curvature.

An alternative and particularly preferred arrangement of the grooves 60 is schematically illustrated in FIG. 6. Thus, the grooves 60 extend substantially horizontally in their longitudinal extension at least in the region of side wall portions 16a of the accommodating container inner wall 26 in the operating position of the device 1, i.e. in particular in a plane perpendicular to a (virtual) cylinder axis of cylinder casing segments of the side wall portions 16a. The horizontal course of the grooves allows targeted lighting only in the region of the filling, i.e. up to the fill level, even if the fill level inside the container varies (or if the single-use container is of different heights). This prevents drying out and burning in of cell material adhering to the single-use container above the liquid level. In this variant, the accommodating container inner wall can also be prefabricated partially with the grooves and then be deformed according to the desired cylinder curvature. This has the additional advantage that the accommodating container inner wall, which has been prefabricated with the grooves, can then be bent to be round without causing uncontrolled or angular deformations (e.g. along the course of the grooves).

1. A device for accommodating a single-use container, comprising:
an accommodating container with an accommodating container inner wall, which delimits a container interior of the accommodating container for accommodating the single-use container,
wherein a plurality of grooves for accommodating electrical functional elements are provided in the accommodating container inner wall.

2. The device according to claim 1, wherein lighting units for illuminating the container interior are at least partially mounted in the plurality of grooves.

3. The device according to claim 2, wherein the grooves are closed off from the container interior using groove covers such that inner surfaces of the groove covers together with inner surfaces of the accommodating container inner wall formed between the grooves form continuous surfaces.

4. The device according to claim 3, wherein an air gap is formed between the lighting units and the respective groove cover.

5. The device according to claim 3, wherein each of the plurality of grooves has a two-step cross-section on both sides perpendicular to a longitudinal axis of the respective groove such that on both sides of a central groove channel, a support surface is set back by a first step relative to the inner surface of the accommodating container inner wall and a groove base of the central groove channel is set back by a second step relative to the two support surfaces.

6. The device according to claim 5, wherein a height of the first step is in a range from about 0.5 mm to about 5 mm; and/or
wherein the height of the second step is in a range from about 2 mm to about 15 mm; and/or
wherein a width of the respective support surface is in a range from about 1 mm to about 5 mm.

7. The device according claim 2, wherein the lighting units that are mountable or mounted in the grooves are designed to emit light substantially within a wavelength range of about 50 nm to about 50 μm.

8. The device according to claim 1, wherein a plurality of grooves in the accommodating container inner wall are arranged in a ring shape and/or parallel to one another, and extending horizontally in an operating position of the device.

9. The device according to claim 1, wherein a plurality of the grooves extend in a straight line and/or parallel to one another and are arranged equidistant from one another.

10. The device according to claim 1, wherein the accommodating container inner wall at least partially forms a part of a cylinder casing surface or is partially composed of parts of cylinder casing surfaces, and wherein the grooves are arranged in one or in each such part of a cylinder casing surface parallel to a respective cylinder axis.

11. The device according to claim 1, wherein the container interior has a volume in the range of about 1 liter to about 5000 liters.

12. The device according to claim 1, wherein the accommodating container inner wall at least partially comprises metal or is formed from metal.

13. The device according to claim 1, which also comprises a temperature control unit for temperature control of the accommodating container inner wall.

14. The device according to claim 13, wherein a maximum cooling capacity of at least about 5 kW can be achieved by the temperature control unit.

15. A use of a device claim 1 for processing phototrophic organisms.

16. The device according to claim 2, wherein the lighting units are LEDs and the lighting units are at least partially covered or cast in the grooves by a cast material.

17. The device according to claim 6, wherein a height of the first step is in a range from about 1 mm to about 3 mm; and/or
wherein the height of the second step is in a range from about 2 mm to about 10 mm; and/or
wherein a width of the respective support surface is in a range from about 1 mm to about 3 mm.

18. The device according to claim 13, wherein a maximum cooling capacity of at least about 10 KW can be achieved by the temperature control unit.

19. The device according to claim 13, wherein a maximum cooling capacity of at least about 15 kW can be achieved by the temperature control unit.

20. The device according to claim 13, wherein a maximum cooling capacity of at least about 20 kW can be achieved by the temperature control unit.
